(19) United States
(12) Patent Application Publication
(10) Pub. No.: US 2010/0168694 A1
(43) Pub. Date: Jul. 1, 2010

(54) Infrared Wetness Detection System For An Absorbent Article

(76) Inventors: Sudhanshu Gakhar, Neenah, WI (US); Sridhar Ranganathan, Suwanee, GA (US); Thomas Michael Ales, III, Neenah, WI (US); Randall George Sarkis, Chaska, MN (US)

Correspondence Address: KIMBERLY-CLARK WORLDWIDE, INC.;Tara Pohlkotte, 2300 Winchester Rd., NEENAH, WI, 54956, US

(21) Appl. No.: 12/636,888

(22) Filed: Dec. 14, 2009

Related U.S. Application Data
(63) Continuation-in-part of application No. 12/347,539, filed on Dec. 31, 2008.

(51) Int. Cl. A61F 13/42; G08B 21/00

(52) U.S. Cl. 604/361; 340/604

(57) ABSTRACT

A signaling device for sensing and indicating the presence of a body exudate in an absorbent article includes a housing and a paired IR generator/detector disposed within the housing, the paired IR generator/detector adapted to sense a change in infrared light reflectance due to an insult to the absorbent article, the paired IR generator/detector including an infrared-generating light-emitting diode and an infrared-detecting phototransistor. In addition, a body exudate collection and detection system includes an absorbent article and a signaling device adapted to be used in conjunction with the absorbent article for detecting the presence of a body exudate therein, the signaling device including a housing and a paired IR generator/detector disposed within the housing, the paired IR generator/detector including an infrared-generating light-emitting diode and an infrared-detecting phototransistor.

### RELATED APPLICATION

The present application is a continuation-in-part of U.S. patent application Ser. No. 12/347,539, filed on Dec. 31, 2008, entitled “Remote Detection Systems for Absorbent Articles.”

### BACKGROUND

Absorbent articles such as diapers, training pants, incontinence products, feminine hygiene products, swim undergarments, and the like, conventionally include a liquid permeable body-side liner, a liquid impermeable outer cover, and an absorbent structure. The absorbent structure is typically located between the outer cover and the liner for taking in and retaining liquids (e.g., urine) exuded by the wearer. The absorbent structure can be made of, for instance, superabsorbent particles. Many absorbent articles, especially those sold under the trade name HUGGIES by the Kimberly-Clark Corporation, are so efficient at absorbing liquids that it is sometimes difficult to tell whether or not the absorbent article has been insulted with a body exudate, especially when the absorbent article is being worn by a newborn or other very young wearers. Insult amounts in such wearers tend to be very small. Other wearers might also produce very small insults.

Accordingly, various types of moisture or wetness indicators have been suggested for use in absorbent articles. The wetness indicators include various passive indicators such as indicator strips, printing, or other devices within each absorbent article, requiring a caregiver to pay for the wetness indicator in each absorbent article whether or not the caregiver intends to use the wetness indicator. Wetness indicators can also include alarm devices that are designed to assist parents or attendants in identifying a wet diaper condition early on. The devices can produce an audible, tactile, electromagnetic, or visual signal. Many of these devices rely on electronics, including conductive elements within each absorbent article that can increase the expense of the absorbent article.

In some aspects, for instance, conductive threads or foils have been placed in the absorbent articles that extend from the front of the article to the back of the article. The conductive materials serve as conductive leads for a signaling device and form an open circuit in the article that can be closed when a body fluid, such as urine, closes the circuit.

Incorporating conductive leads into absorbent articles, however, has caused various problems. For example, absorbent articles are typically mass produced on very fast moving machinery. Incorporating conductive leads into an absorbent article at conventional machine speeds has been problematic.

In addition, packaged absorbent articles are typically fed through a metal detector to ensure that there are no foreign objects contained in the package. If the conductive leads are made from or contain a metal, the metal detector can be activated registering a false positive. The incorporation of metallic materials into absorbent articles can also cause problems for those wearing the garments when attempting to pass through security gates that include metal detectors.

In view of the above, a need currently exists for a signaling system for an absorbent article that does not require conductive elements containing metal or other devices to be inserted into the interior of the article.

### SUMMARY

The present inventors undertook intensive research and development efforts with respect to improving absorbent articles, particularly in providing a wetness indicator only when desired by a caregiver and without adding to the cost of an absorbent article. A need exists for wetness detection in diapers and incontinence products in general. Technology that can be implemented without altering diaper construction is preferred.

A non-invasive sensor measures infrared reflectance at some depth within an absorbent article. A useful approach is a paired IR generator/detector system that can be attached to an appropriate target zone on the outer cover of the absorbent article.

The present disclosure is generally directed to various signaling systems that are particularly well suited for use in conjunction with absorbent articles. The signaling systems, for instance, can be connected to a signaling device that can be configured to emit a signal, such as an audible, tactile, electromagnetic or visual signal, for indicating to a user that a body fluid is present in the absorbent article. For example, in one aspect, the absorbent article includes a diaper and the signaling system is configured to indicate the presence of urine or a bowel movement. In other absorbent articles, however, the signaling systems can be configured to indicate the presence of yeast or metabolites.

More particularly, the present disclosure is directed to signaling systems for absorbent articles that can detect the presence of a body fluid without having to place or insert conductive elements into the interior of the article. For instance, in one aspect, a sensor can be mounted to an exterior surface of the absorbent article that is capable of sensing a change on the interior of the article that indicates the presence of a body fluid, such as urine, which is an infrared-absorbing fluid. In this aspect, the sensor can include, for instance, a paired IR generator/detector system. Insulting the absorbent article with urine will create a change in infrared reflectance as some of the infrared is absorbed by the insult. The sensor can be placed in communication with a signaling device. Once a change within the interior of the absorbent article is detected, the signaling device can be configured to emit a signal that indicates a body fluid is present in the absorbent article.

The present disclosure is directed to a signaling device for sensing and indicating the presence of a body exudate in an absorbent article, the device including a housing and a paired IR generator/detector disposed within the housing, the paired IR generator/detector adapted to sense a change in infrared light reflectance due to an insult to the absorbent article, the paired IR generator/detector including an infrared-generating light-emitting diode and an infrared-detecting phototransistor.

The present disclosure is also directed to a body exudate collection and detection system including an absorbent article and a signaling device adapted to be used in conjunction with the absorbent article for detecting the presence of a body exudate therein, the signaling device including a housing and a paired IR generator/detector disposed within the housing, the paired IR generator/detector including an infrared-generating light-emitting diode and an infrared-detecting phototransistor.

The present disclosure is also directed to a signaling device for sensing and indicating the presence of a body exudate in an absorbent article, the device including a housing and an attachment mechanism for removably attaching the housing to the absorbent article. The device also includes a paired IR generator/detector disposed within the housing, the paired IR generator/detector including an infrared-generating light-emitting diode and an infrared-detecting phototransistor, wherein the signaling device is adapted to emit a signal when an insult within the absorbent structure is sensed by the paired IR generator/detector.

Other features and aspects of the present disclosure are discussed in greater detail herein.

### BRIEF DESCRIPTION OF THE DRAWINGS

The foregoing and other features and aspects of the present disclosure and the manner of attaining them will become more apparent, and the disclosure itself will be better understood by reference to the following description, appended claims, and accompanying drawings.

FIG. 1 is a rear perspective view of one aspect of an absorbent article;

FIG. 2 is a front perspective view of the absorbent article illustrated in FIG. 1 including one aspect of a wetness indicator of the present disclosure;

FIG. 3 is a plan view of the absorbent article shown in FIG. 1 with the article in an unfastened, unfolded and laid flat condition showing the surface of the article that faces away from the wearer;

FIG. 4 is a plan view similar to FIG. 3 showing the surface of the absorbent article that faces the wearer when worn and with portions cut away to show underlying features;

FIG. 5 is a block diagram of a signaling device of the present disclosure; and

FIG. 6 is the sensor transient response to repeated insults using the signaling device and absorbent article of the present disclosure.

Repeat use of reference characters in the present specification and drawings is intended to represent the same or analogous features or elements of the present disclosure.

### DETAILED DESCRIPTION

It is to be understood by one of ordinary skill in the art that the present discussion is a description of exemplary aspects only, and is not intended as limiting the broader aspects of the present disclosure.

The present disclosure is generally directed to signaling systems for absorbent articles that indicate to a user when a body fluid has insulted the article. For example, in one aspect, the signaling system is designed to emit a signal when urine is detected in the absorbent article. The absorbent article can be, for instance, a diaper, a training pant, an incontinence product, a feminine hygiene product, a medical garment, a bandage, and the like.

Of particular advantage, signaling systems made in accordance with the present disclosure can sense the presence of a body fluid within the absorbent article without having to construct the absorbent article with any elements or sensors contained in the interior of the article. In the past, for instance, metallic conductive leads were typically placed within the interior of the absorbent article. The signaling systems of the present disclosure, on the other hand, can sense the presence of a body fluid from an exterior surface of the article that can greatly simplify the incorporation of the signaling system into the article.

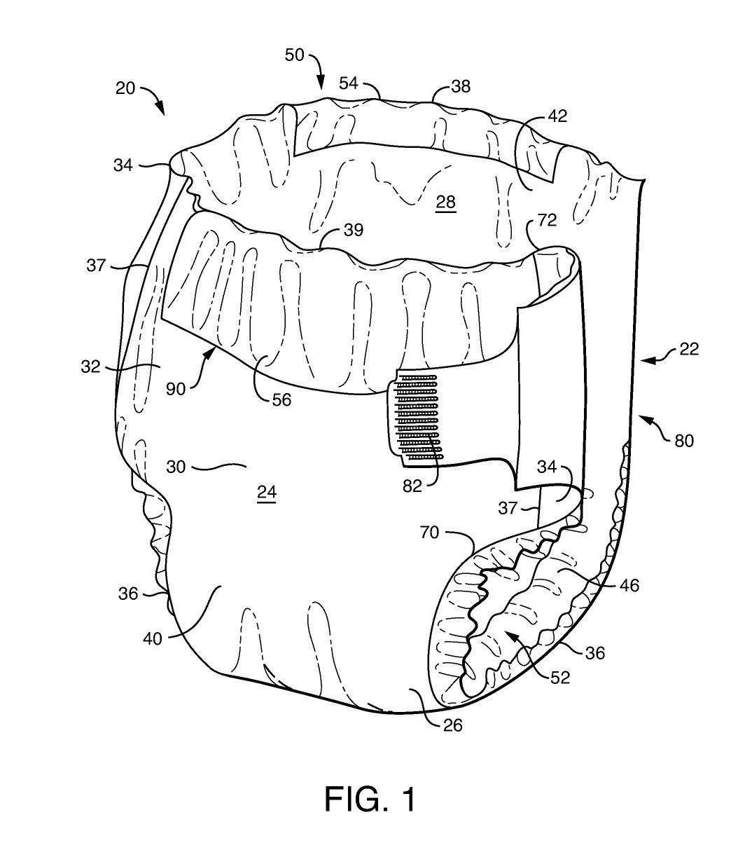

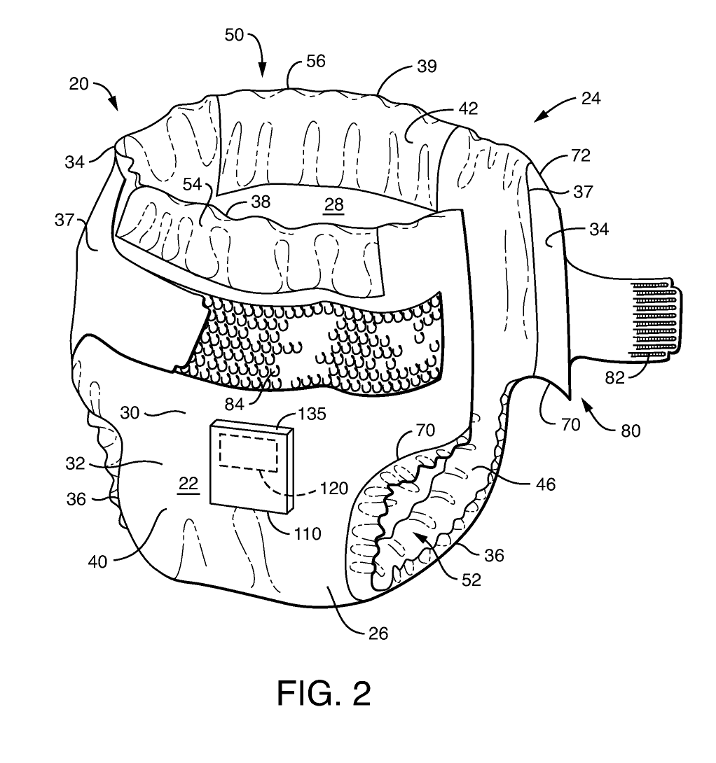

In accordance with the present disclosure, the signaling system can have various configurations and designs. Referring to FIGS. 1 and 2, for exemplary purposes, an absorbent article 20 that can be used in conjunction with signaling systems of the present disclosure is shown. The absorbent article 20 can be disposable or not. It is understood that the present disclosure is suitable for use with various other absorbent articles intended for personal wear, including but not limited to diapers, training pants, swim pants, feminine hygiene products, incontinence products, medical garments, surgical pads and bandages, other personal care or health care garments, and the like without departing from the scope of the present disclosure.

By way of illustration only, various materials and methods for constructing absorbent articles such as the diaper 20 of the various aspects of the present disclosure are disclosed in PCT Patent Application WO 00/37009 published Jun. 29, 2000 by A. Fletcher et al; U.S. Pat. No. 4,940,464 issued Jul. 10, 1990 to Van Gompel et al.; U.S. Pat. No. 5,766,389 issued Jun. 16, 1998 to Brandon et al., and U.S. Pat. No. 6,645,190 issued Nov. 11, 2003 to Olson et al. which are incorporated herein by reference to the extent they are consistent (i.e., not in conflict) herewith.

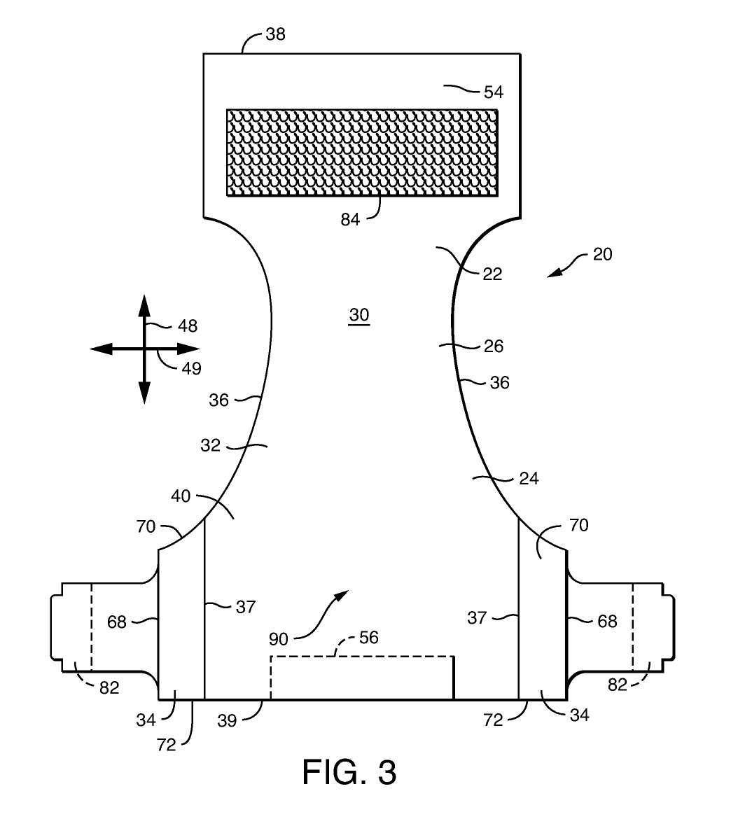

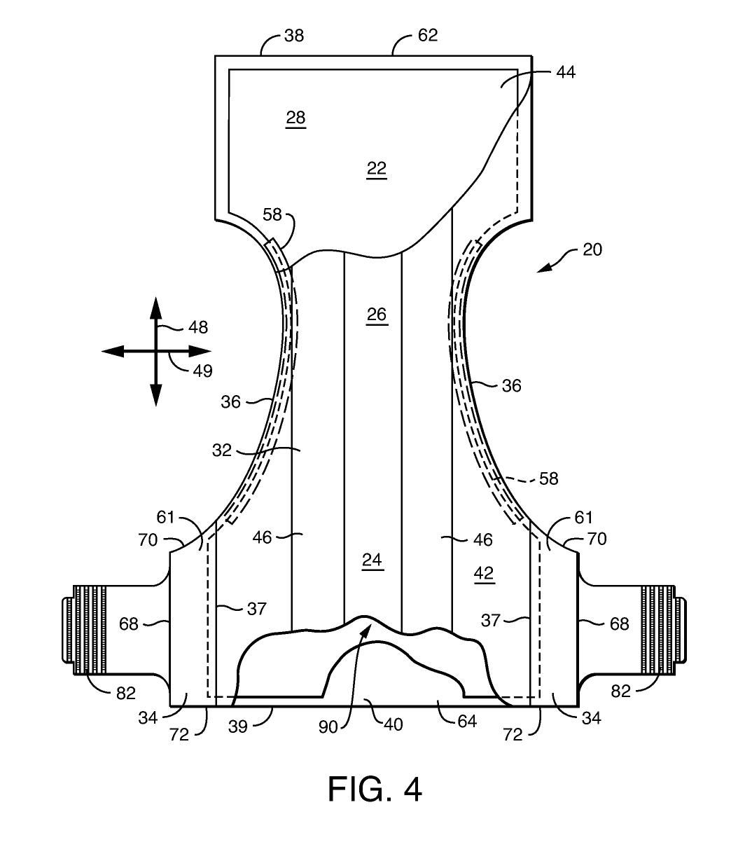

A diaper 20 is representatively illustrated in FIG. 1 in a partially fastened condition. The diaper 20 shown in FIGS. 1 and 2 is also represented in FIGS. 3 and 4 in an opened and unfolded state. Specifically, FIG. 3 is a plan view illustrating the exterior side of the diaper 20, while FIG. 4 illustrates the interior side of the diaper 20. As shown in FIGS. 3 and 4, the diaper 20 defines a longitudinal direction 48 that extends from the front of the article when worn to the back of the article. Orthogonal to the longitudinal direction 48 is a lateral direction 49.

The diaper 20 defines a pair of longitudinal end regions, otherwise referred to herein as a front region 22 and a back region 24, and a center region, otherwise referred to herein as a crotch region 26, extending longitudinally between and interconnecting the front and back regions 22, 24. The diaper 20 also defines an inner surface 28 adapted in use (e.g., positioned relative to the other components of the article 20) to be disposed toward the wearer, and an outer surface 30 opposite the inner surface. The front and back regions 22, 24 are those portions of the diaper 20, which, when worn, wholly or partially cover or encircle the waist or mid-lower torso of the wearer. The crotch region 26 generally is that portion of the diaper 20 which, when worn, is positioned between the legs of the wearer and covers the lower torso and crotch of the wearer. The absorbent article 20 has a pair of laterally opposite side edges 36 and a pair of longitudinally opposite waist edges, respectively designated front waist edge 38 and back waist edge 39.

The illustrated diaper 20 includes a chassis 32 that, in this aspect, encompasses the front region 22, the back region 24, and the crotch region 26. Referring to FIGS. 1-4, the chassis 32 includes an outer cover 40 and a bodyside liner 42 (FIGS. 1 and 4) that can be joined to the outer cover 40 in a superimposed relation therewith by adhesives, ultrasonic bonds, thermal bonds or other conventional techniques. Referring to FIG. 4, the liner 42 can suitably be joined to the outer cover 40 along the perimeter of the chassis 32 to form a front waist seam 62 and a back waist seam 64. As shown in FIG. 4, the liner 42 can suitably be joined to the outer cover 40 to form a pair of side seams 61 in the front region 22 and the back region 24. The liner 42 can be generally adapted, i.e., positioned relative to the other components of the article 20, to be disposed toward the wearer's skin during wear of the absorbent article. The chassis 32 can further include an absorbent structure 44 particularly shown in FIG. 4 disposed between the outer cover 40 and the bodyside liner 42 for absorbing liquid body exudates exuded by the wearer, and can further include a pair of containment flaps 46 secured to the bodyside liner 42 for inhibiting the lateral flow of body exudates.

The elasticized containment flaps 46 as shown in FIG. 4 define a partially unattached edge that assumes an upright configuration in at least the crotch region 26 of the diaper 20 to form a seal against the wearer's body. The containment flaps 46 can extend longitudinally along the entire length of the chassis 32 or can extend only partially along the length of the chassis. Suitable constructions and arrangements for the containment flaps 46 are generally well known to those skilled in the art and are described in U.S. Pat. No. 4,704,116 issued Nov. 3, 1987 to Enloe, which is incorporated herein by reference.

To further enhance containment and/or absorption of body exudates, the diaper 20 can also suitably include leg elastic members 58 (FIG. 4), as are known to those skilled in the art. The leg elastic members 58 can be operatively joined to the outer cover 40 and/or the bodyside liner 42 and positioned in the crotch region 26 of the absorbent article 20.

The leg elastic members 58 can be formed of any suitable elastic material.

As is well known to those skilled in the art, suitable elastic materials include sheets, strands or ribbons of natural rubber, synthetic rubber, or thermoplastic elastomeric polymers. The elastic materials can be stretched and adhered to a substrate, adhered to a gathered substrate, or adhered to a substrate and then elasticized or shrunk, for example with the application of heat, such that elastic retractive forces are imparted to the substrate. In one particular aspect, for example, the leg elastic members 58 can include a plurality of dry-spun coalesced multifilament spandex elastomeric threads sold under the trade name LYCRA and available from Invista, Wilmington, Del., U.S.A.

In some aspects, the absorbent article 20 can further include a surge management layer (not shown) that can be optionally located adjacent the absorbent structure 44 and attached to various components in the article 20 such as the absorbent structure 44 or the bodyside liner 42 by methods known in the art, such as by using an adhesive. A surge management layer helps to decelerate and diffuse surges or gushes of liquid that can be rapidly introduced into the absorbent structure of the article. Desirably, the surge management layer can rapidly accept and temporarily hold the liquid prior to releasing the liquid into the storage or retention portions of the absorbent structure. Examples of suitable surge management layers are described in U.S. Pat. No. 5,486,166; and U.S. Pat. No. 5,490,846. Other suitable surge management materials are described in U.S. Pat. No. 5,820,973. The entire disclosures of these patents are hereby incorporated by reference herein to the extent they are consistent (i.e., not in conflict) herewith.

As shown in FIGS. 1-4, the absorbent article 20 further includes a pair of opposing elastic side panels 34 that are attached to the back region of the chassis 32. As shown particularly in FIGS. 1 and 2, the side panels 34 can be stretched around the waist and/or hips of a wearer to secure the garment in place. As shown in FIGS. 3 and 4, the elastic side panels are attached to the chassis along a pair of opposing longitudinal edges 37. The side panels 34 can be attached or bonded to the chassis 32 using any suitable bonding technique. For instance, the side panels 34 can be joined to the chassis by adhesives, ultrasonic bonds, thermal bonds, or other conventional techniques.

In an alternative aspect, the elastic side panels can also be integrally formed with the chassis 32. For instance, the side panels 34 can include an extension of the bodyside liner 42, of the outer cover 40, or of both the bodyside liner 42 and the outer cover 40.

In the aspects shown in the figures, the side panels 34 are connected to the back region of the absorbent article 20 and extend over the front region of the article when securing the article in place on a user. It should be understood, however, that the side panels 34 can alternatively be connected to the front region of the article 20 and extend over the back region when the article is donned.

With the absorbent article 20 in the fastened position as partially illustrated in FIGS. 1 and 2, the elastic side panels 34 can be connected by a fastening system 80 to define a 3-dimensional diaper configuration having a waist opening 50 and a pair of leg openings 52. The waist opening 50 of the article 20 is defined by the waist edges 38 and 39 that encircle the waist of the wearer.

In the aspects shown in the figures, the side panels are releasably attachable to the front region 22 of the article 20 by the fastening system. It should be understood, however, that in other aspects the side panels can be permanently joined to the chassis 32 at each end. The side panels can be permanently bonded together, for instance, when forming a training pant or absorbent swimwear.

The elastic side panels 34 each have a longitudinal outer edge 68, a leg end edge 70 disposed toward the longitudinal center of the diaper 20, and waist end edges 72 disposed toward a longitudinal end of the absorbent article. The leg end edges 70 of the absorbent article 20 can be suitably curved and/or angled relative to the lateral direction 49 to provide a better fit around the wearer's legs. However, it is understood that only one of the leg end edges 70 can be curved or angled, such as the leg end edge of the back region 24, or alternatively, neither of the leg end edges can be curved or angled, without departing from the scope of the present disclosure. As shown in FIG. 4, the outer edges 68 are generally parallel to the longitudinal direction 48 while the waist end edges 72 are generally parallel to the transverse axis 49. It should be understood, however, that in other aspects the outer edges 68 and/or the waist edges 72 can be slanted or curved as desired. Ultimately, the side panels 34 are generally aligned with a waist region 90 of the chassis.

The fastening system 80 can include laterally opposite first fastening components 82 adapted for refastenable engagement to corresponding second fastening components 84. In the aspect shown in the figures, the first fastening component 82 is located on the elastic side panels 34, while the second fastening component 84 is located on the front region 22 of the chassis 32. In one aspect, a front or outer surface of each of the fastening components 82, 84 includes a plurality of engaging elements. The engaging elements of the first fastening components 82 are adapted to repeatedly engage and disengage corresponding engaging elements of the second fastening components 84 to releasably secure the article 20 in its three-dimensional configuration.

The fastening components 82, 84 can be any refastenable fasteners suitable for absorbent articles, such as adhesive fasteners, cohesive fasteners, mechanical fasteners, or the like. In particular aspects the fastening components include mechanical fastening elements for improved performance. Suitable mechanical fastening elements can be provided by interlocking geometric shaped materials, such as hooks, loops, bulbs, mushrooms, arrowheads, balls on stems, male and female mating components, buckles, snaps, or the like.

In the illustrated aspect, the first fastening components 82 include hook fasteners and the second fastening components 84 include complementary loop fasteners. Alternatively, the first fastening components 82 can include loop fasteners and the second fastening components 84 can be complementary hook fasteners. In another aspect, the fastening components 82, 84 can be interlocking similar surface fasteners, or adhesive and cohesive fastening elements such as an adhesive fastener and an adhesive-receptive landing zone or material; or the like.

In addition to possibly having elastic side panels, the absorbent article 20 can include various waist elastic members for providing elasticity around the waist opening. For example, as shown in the figures, the absorbent article 20 can include a front waist elastic member 54 and/or a back waist elastic member 56.

In one aspect of the present disclosure best illustrated in FIG. 2, a signaling device 110 includes a paired IR generator/detector 120 that is adapted to detect the presence of a body exudate in the absorbent article 20. The paired IR generator/detector 120 includes an infrared-generating light-emitting diode (LED) and an infrared-detecting photo-transistor.

Optics technology such as that described herein can be used to detect wetness in an absorbent article. Light attenuates in water and is more readily absorbed by a wet article as opposed to a dry one. This absorption of light by water is especially significant in the near-infrared region of 800 to 980 nanometer wavelengths. A paired IR generator/detector 120, particularly one tuned to this same wavelength of infrared light, can be used for wetness detection and indication in an absorbent article. The paired IR generator/detector 120, and the components therein, can be selected to be tuned with one or more body exudates including urine.

Light emitted by an infrared-generating LED is reflected off a surface close to the sensor system and is collected by the infrared-detecting phototransistor positioned adjacent to the infrared-generating LED. The electrical current through the infrared-detecting phototransistor is detected by a voltage drop across a sensing resistor and is proportional to the intensity of the received infrared signal.

When the product is dry, most of the infrared light emitted by the infrared-generating LED is reflected off various components of the absorbent article 20 and is collected by the infrared-detecting phototransistor. This can result, for example, in a full 5 volt output. After the absorbent article 20 has received an insult, more infrared from the infrared-generating LED is absorbed by the insulted absorbent article 20 than was absorbed by the absorbent article 20 when the absorbent article 20 was dry. This greater absorption of infrared light results in less reflectance of infrared light from the absorbent article 20. In some cases, the amount of infrared light from the infrared-generating LED absorbed by the insulted absorbent article 20 can be greater than the amount of infrared light reflected by the insulted absorbent article 20. The reduced amount of infrared light received by the infrared-detecting phototransistor results in a reduction in voltage drop across the sensing resistor.

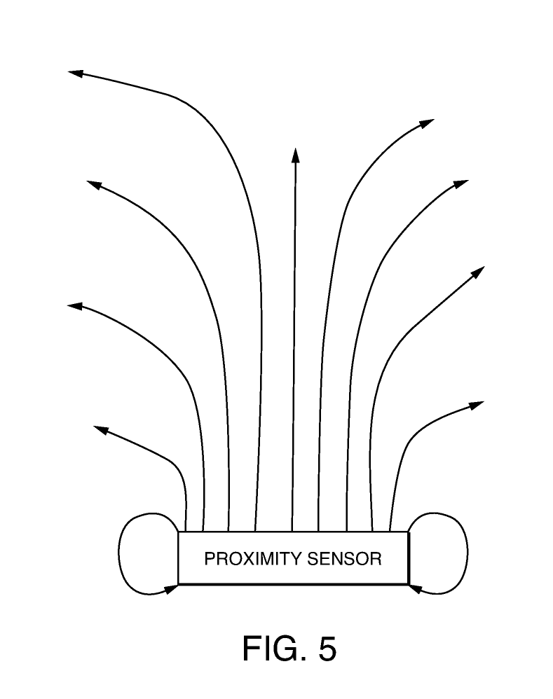

In other aspects of the present disclosure, one or more paired IR generator/detectors 120 can be used in conjunction with the absorbent article 20. A block diagram of such a system is shown in FIG. 5.

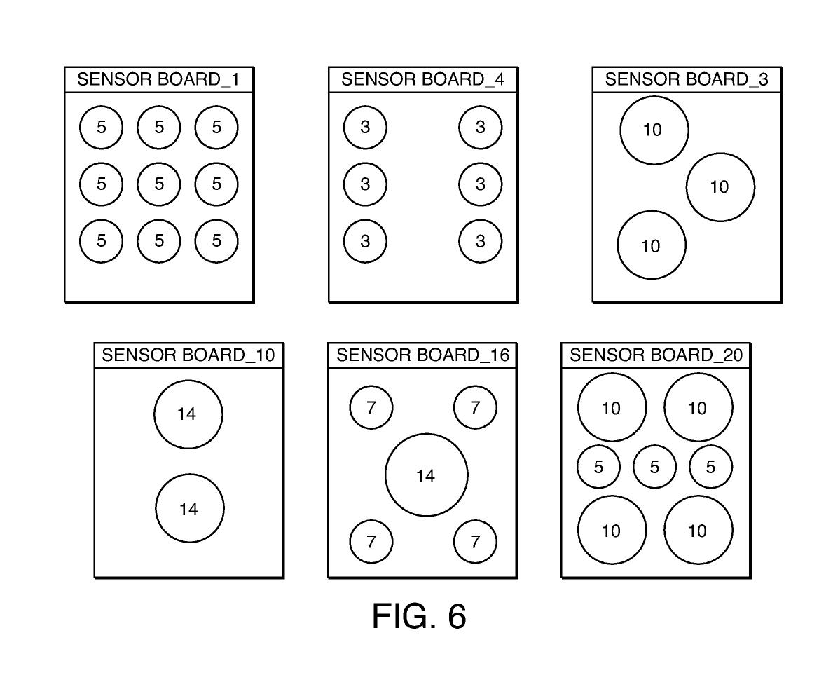

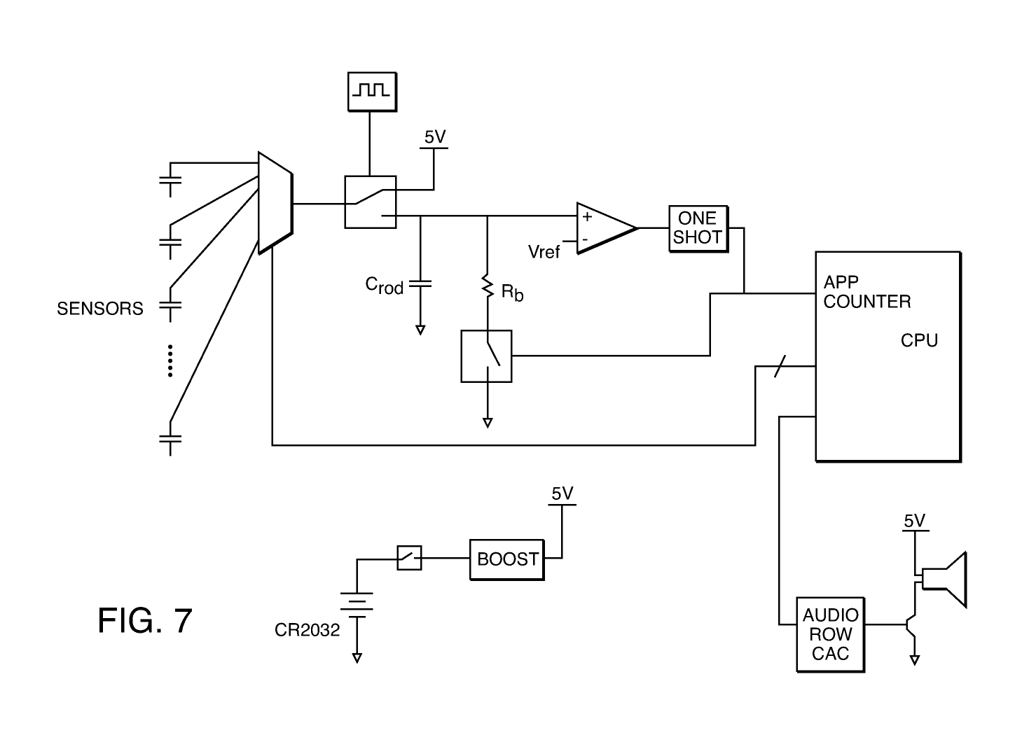

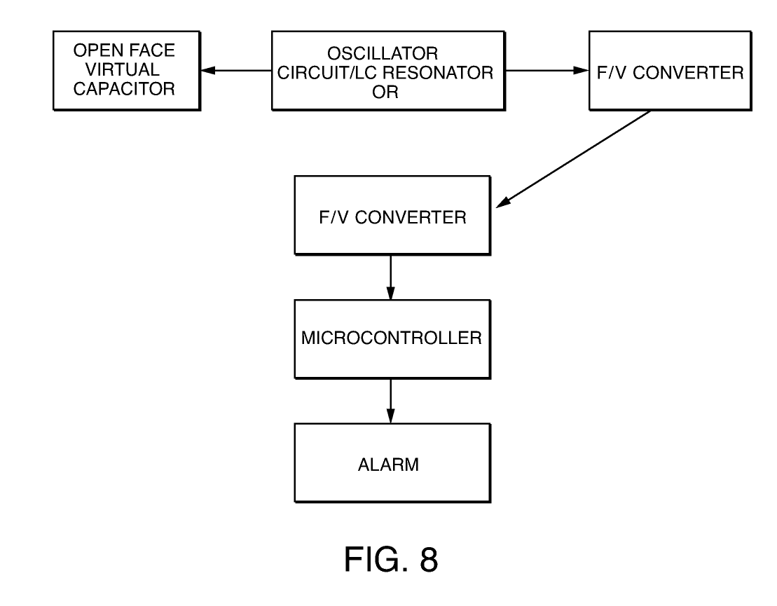

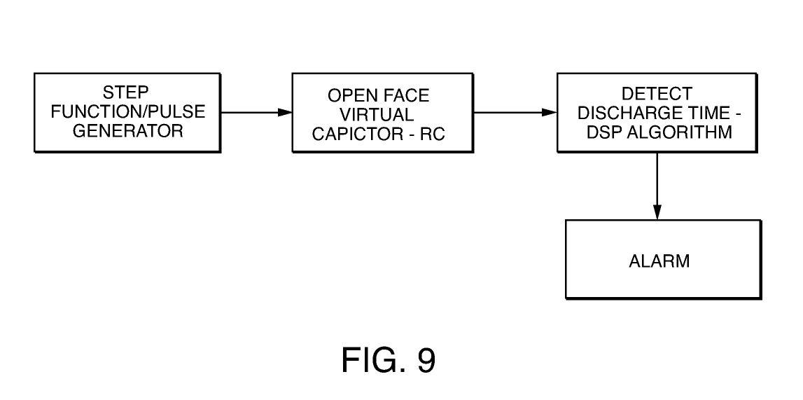

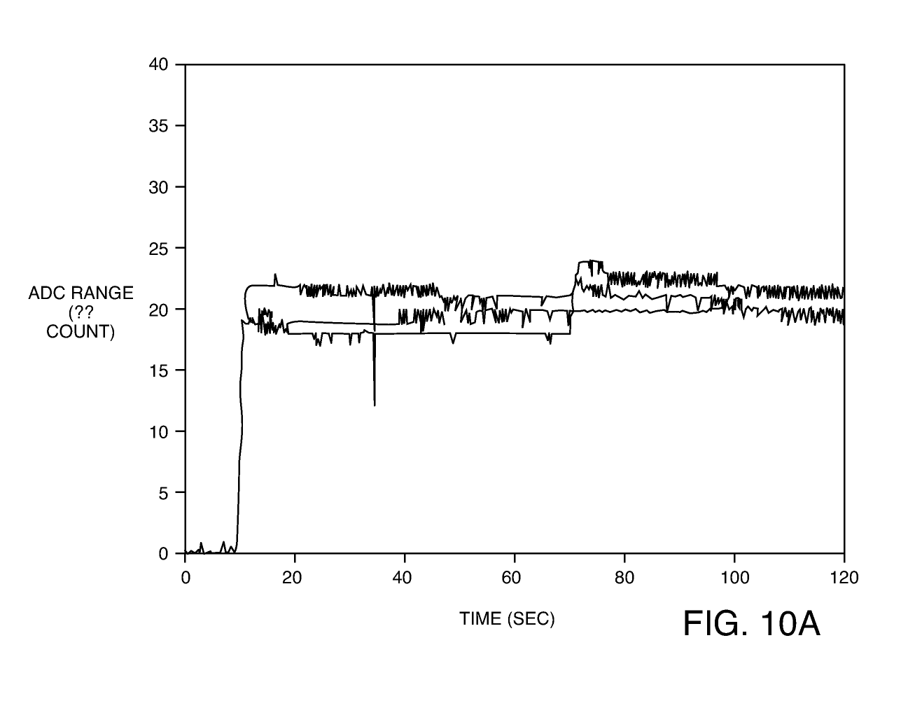

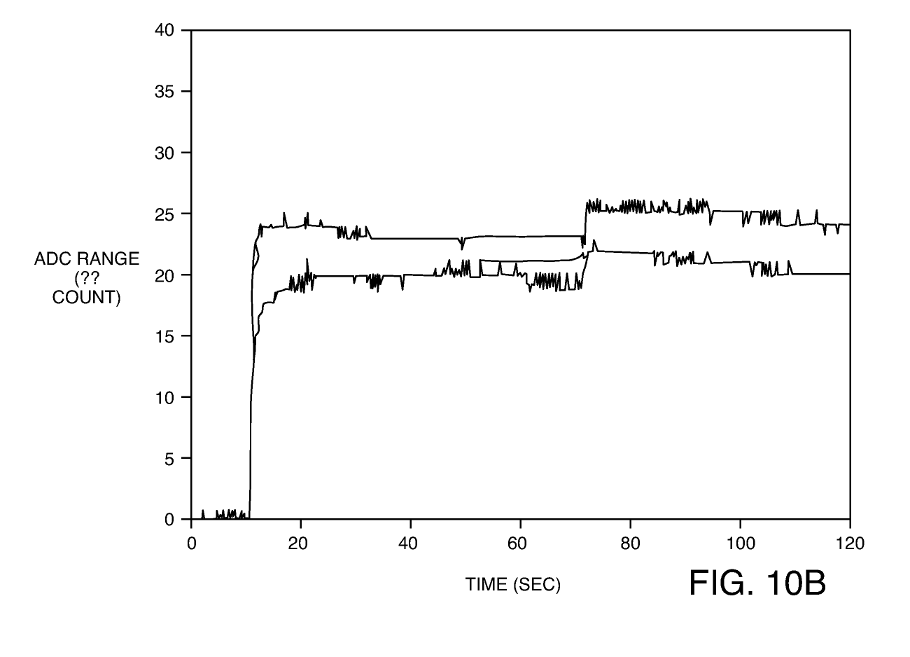

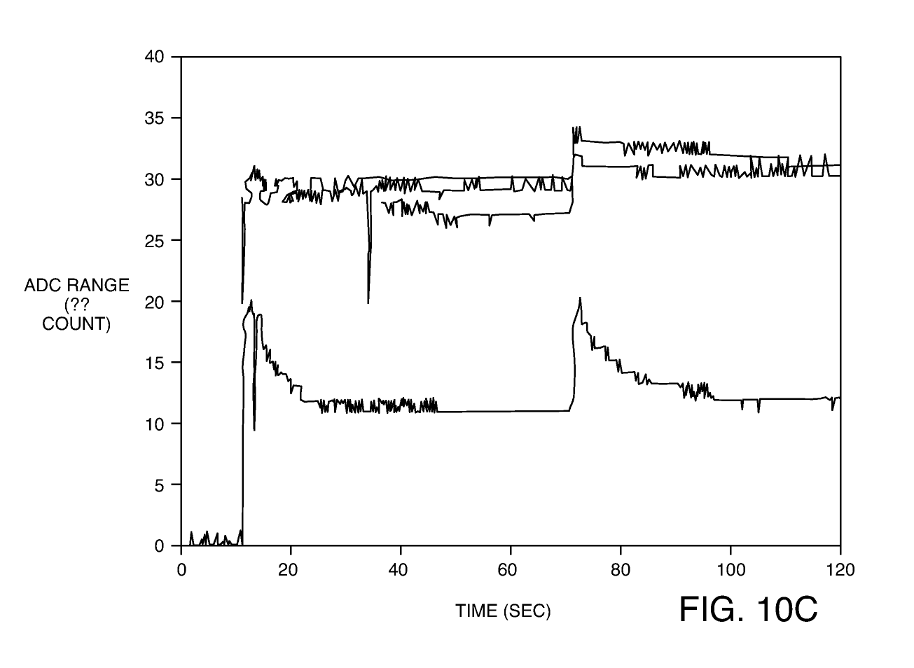

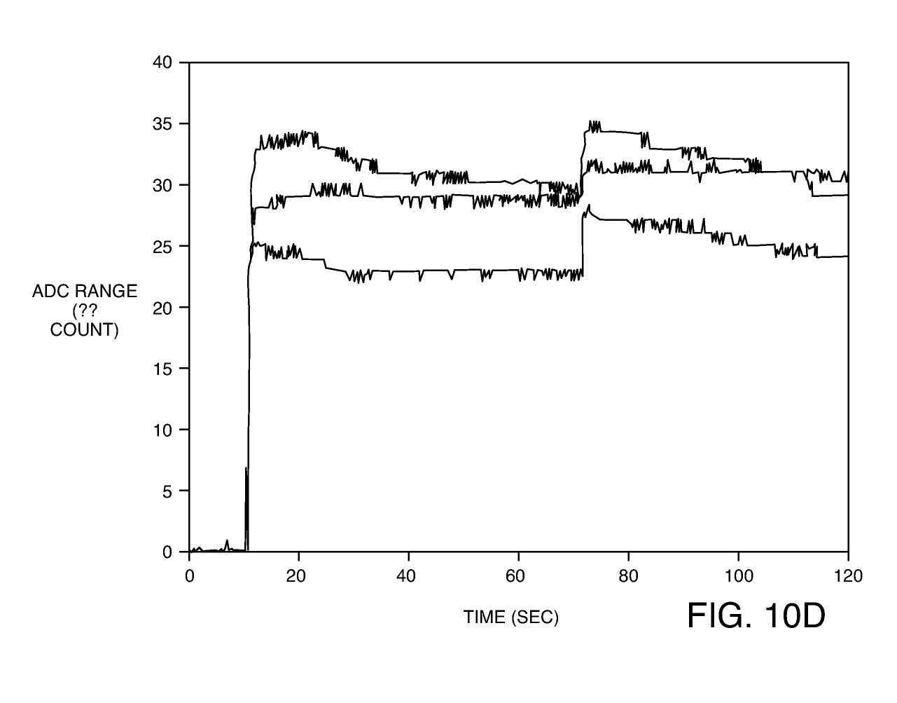

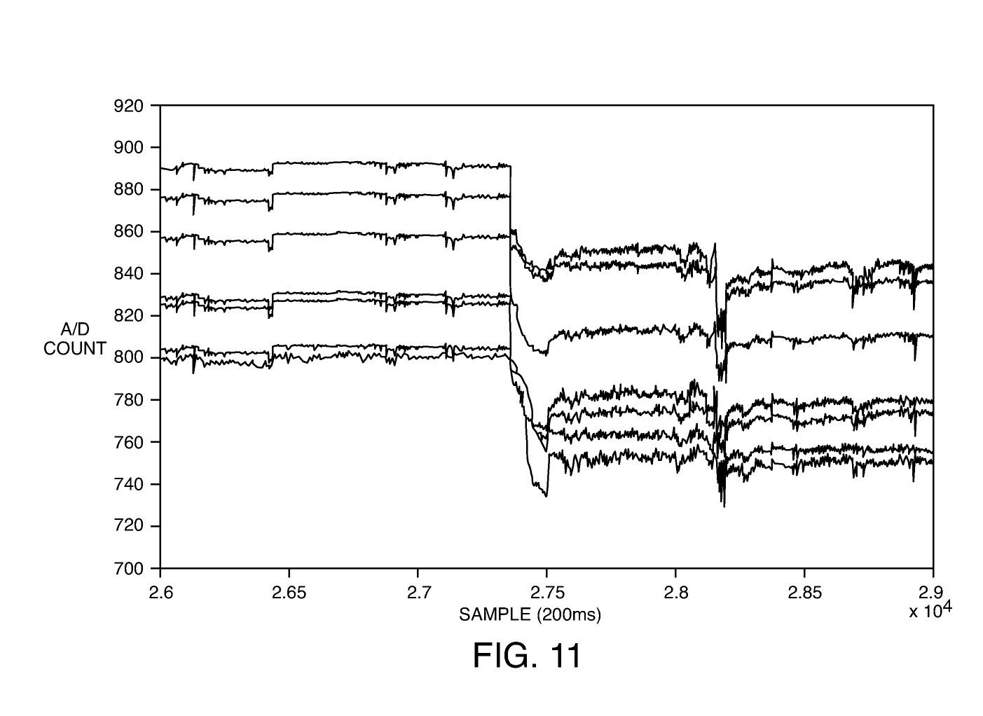

Experimental data proving the concept is shown in FIG. 6. An absorbent article 20 of the present disclosure was laid open and flat on top of a standard circuit using a paired IR generator/detector 120 of the present disclosure. The absorbent article 20 was insulted with 10 ml of saline after 90 seconds, with an additional 30 ml of saline after 180 seconds, and with an additional 30 ml of saline after 270 seconds. The voltage output of the paired IR generator/detector 120 was measured and plotted in FIG. 6. Further experimentation with such a system also indicated a quick response to wetness in the absorbent product (<3 sec) and demonstrated a capability of detecting multiple small voids on the order of 10 ml.

In alternate aspects of the present disclosure, similar sensor arrangements can be used with such sensor arrangements using visible, ultraviolet, or other infrared wavelengths.

Infrared LEDs are preferable as an infrared-generating source because of their low power requirements, allowing for longer life for a given battery or other power source. Suitable paired IR generator/detectors such as an AVAGO Technologies HSDL—9100 Surface-Mount Proximity Sensor are available from Avago Inc. Additional battery or other power source life can be attained through power use optimization and algorithm development as is known in the art.

Use of the paired IR generator/detector 120 described herein enables a conductor-less wetness sensing solution for absorbent articles that has no impact on current absorbent article manufacturing processes because the paired IR generator/detector is not manufactured as a part of the absorbent article 20, and because the absorbent article 20 has no conductors positioned therein. The paired IR generator/detector has a quick response time, generally less than 3 seconds, is capable of detecting small voids such as those less than 10 milliliters, and can detect multiple insults. The paired IR generator/detector 120 can be sufficiently powered using a commonly-available battery such as a 2032 Li Battery for a battery life of at least three months. Use of the paired IR generator/detector 120 with its common components also allows the device to meet regulatory standards in the United States and in the European Union.

For example, a noninvasive, paired IR generator/detector 120 can be used to determine the IR reflectance of material near the sensing element. As described herein, this technology can be applied to detect wetness in an absorbent article 20 from outside the outer cover 40. Key challenges, however, with such a paired IR generator/detector 120 are managing the distance of the target from the paired IR generator/detector 120 to have sufficient distance for reflection, and the ability of the system to detect a small amount of wetness from outside the outer cover. Such challenges can be addressed through signal conditioning and algorithms to ignore environmental interferences.

When insult conditions are reached, as determined by the algorithm running the microcontroller such as a PIC 16F876A, an alarm signal is generated as further described herein.

The signaling device 110 can emit any suitable signal to indicate to the user that the article has been insulted. The signal, for instance, can include an audible signal, a tactile signal, an electromagnetic signal, or a visual signal. The audible signal, for instance, can be as simple as a beep or can include a musical tune. In still another aspect, the signaling device can emit a wireless signal that then activates a remote device, such as a telephone or a pager.

Further aspects of the signaling device 110 can be found in co-pending U.S. patent application Ser. No. 12/347,539, entitled “Remote Detection Systems for Absorbent Articles,” which is incorporated herein by reference to the extent it does not conflict herewith.

The electronics associated with the paired IR generator/detector 120 are relatively simple and can be miniaturized. The complete paired IR generator/detector 120 is disposed in a housing 135 (see FIG. 2) that is adapted to be attached to the absorbent article 20, or held in vicinity to the absorbent article 20. If the housing 135 is to be attached to the absorbent article 20 using an attachment mechanism, the housing 135 can be a pouch or a rigid or semi-rigid housing 135 that attaches to the outer cover 40 of the absorbent article 20 near the region where insults are expected. Such attachment mechanism can use adhesive, hook and loop, mechanical fasteners such as snaps, a clip, or a clasp, any other suitable attachment mechanism, or any combination of these. Various attachment mechanisms include those disclosed in co-pending and co-assigned U.S. Patent Application Publication No. 2007/0142797 to Long, et al. and entitled “Garments With Easy-To-Use Signaling Device”; U.S. Patent Application Publication No. 2006/0244614 to Long and entitled “Connection Mechanisms”; and U.S. Patent Application Publication No. 2007/0024457 to Long, et al. and entitled “Connection Mechanisms In Absorbent Articles For Body Fluid Signaling Devices” which are incorporated herein by reference to the extent they are consistent (i.e., not in conflict) herewith.

In another aspect of the present disclosure, the signaling device 110 is adapted to be held near the outermost surface of the outer cover 40 of the absorbent article 20. In this aspect, no attachment mechanism is needed. The wearer of the absorbent article 20 or a caregiver holds the signaling device 110 near the outer cover 40 of the absorbent article 20 to detect whether the absorbent article 20 has received an insult.

Sensors such as those described herein are further described, for instance, in U.S. patent application Ser. No. 11/511,583 and in U.S. Patent Application Publication No. 2008/0048786, which are both incorporated herein by reference to the extent they do not conflict herewith.

For example, in one aspect, the system can be configured such that the signaling device will not emit signals within a certain period of time once the system is first activated, where being activated means the system is in a condition to detect and provide a signal. The period of time can vary depending upon the particular circumstances and the particular application. For example, in one aspect, the system can be configured not to emit signals for at least the first 15 minutes, such as at least the first 30 minutes, such as at least the first 45 minutes, such as at least the first hour the absorbent article is worn.

In an alternative aspect, steady state is determined by the paired IR generator/detector 120 used in the system. Steady state can be determined when substantial or significant changes in infrared reflectance fail to occur for a certain period of time indicating that steady state conditions have been reached. For instance, the system can be configured to only become activated once the paired IR generator/detector 120 determines no substantial changes within the interior of the article for a period of about five minutes, such as about 10 minutes, such as about 20 minutes, such as about 30 minutes, such as about 45 minutes, such as about one hour. For example, if the sensor is a paired IR generator/detector 120, steady state can be determined when the paired IR generator/detector 120 senses no more than about 5 percent change in infrared reflectance on the interior of the article for a period of at least 10 minutes.

When using a paired IR generator/detector 120, the paired IR generator/detector 120 can be placed in any suitable location on the absorbent article 20. For instance, the paired IR generator/detector 120 can be placed in the crotch region 26, on the back region 24, or on the front region 22 of the article 20 depending upon various factors. As described herein, in certain applications, the paired IR generator/detector 120 can be placed on an exterior surface of the outer cover 40 of the absorbent article 20.

All of the sensors described herein can be configured to be disposed of with the absorbent article 20. When disposable, the paired IR generator/detector 120 can be integrated into the outer cover 40 of the absorbent article 20. For instance, in one aspect, the outer cover 40 can include more than one layer and the paired IR generator/detector 120 can be positioned in between the two layers.

In an alternative aspect, the paired IR generator/detector 120 can be configured to be removed from the absorbent article 20 when the absorbent article 20 is disposed and placed on a new absorbent article 20. In fact, in one aspect, the paired IR generator/detector 120 and/or signal device can include multiple settings depending upon the absorbent article 20 to which it is attached. In this manner, the signaling system can be modified based upon the particular product specifications. The product purchased can provide information to the consumer as to which setting to use.

As absorbent articles increase in effectiveness, in one aspect, the signaling system of the present disclosure can be configured to emit a signal or not emit a signal during a first insult of urine and/or to emit a signal when a second insult occurs. In one aspect, for instance, the absorbent article 20 can be constructed so as to be capable of holding two insults of urine from the wearer. A wetness sensing system can be particularly needed for these types of absorbent articles 20 so that a caregiver can differentiate between the first insult and the second insult. In accordance with the present disclosure, the signaling system can be constructed so as to recognize a change within the absorbent article 20 due to the first insult and then readjust the criteria based upon the second insult. Once the second insult is recognized, the signaling system can be designed to emit a signal.

For instance, after a first insult with urine, the paired IR generator/detector 120 can sense a change in infrared light reflectance within the absorbent article 20. The paired IR generator/detector 120 can also be configured to sense a change in infrared light reflectance after second and succeeding insults as well.

When using a paired IR generator/detector 120 as described herein, in one aspect, the system can be designed to take into account changes in the above measurements when the absorbent article 20 is first placed on the wearer. For example, when the absorbent article 20 is first donned, a change in infrared reflectance can be expected. To account for this change, the system of the present disclosure can be configured to only cause signals to be emitted by the signaling device 110 when steady state conditions within the article have been reached.

In some instances, it is conceivable that the paired IR generator/detector 120 needs to contend with nearby objects that can cause interference. In practical applications, however, such a situation is unlikely because the interference-causing object typically needs to be very close to the paired IR generator/detector 120. This makes the appearance of an interference-causing object unlikely when the paired IR generator/detector 120 is used in conjunction with an absorbent article 20. Nevertheless, an interference problem of this sort can be managed by an intelligent algorithm that recognizes and stores signal output once the paired IR generator/detector 120 is in position and activated. The algorithm uses this signal output as a reference point and interprets subsequent signals in relation to this reference point. In other words, the algorithm includes an intelligent zeroing feature.

Once the device is activated, the algorithm takes a baseline measurement, which is automatic and transparent to the user. Once the signaling device 110 is installed by a user, the paired IR generator/detector 120 automatically zeroes itself to establish the point of zero wetness baseline needed.

In another aspect of the present disclosure (not shown), the signaling device 110 uses more than one paired IR generator/detector 120. For example, two paired IR generator/detectors 120 can be positioned such that one is near the front of the absorbent article 20 to detect urine and the other is near the rear of the absorbent article 20 to detect fecal matter.

These and other modifications and variations to the present disclosure can be practiced by those of ordinary skill in the art, without departing from the spirit and scope of the present disclosure, which is more particularly set forth in the appended claims. In addition, it should be understood that aspects of the various aspects can be interchanged both in whole and in part. Furthermore, those of ordinary skill in the art will appreciate that the foregoing description is by way of example only, and is not intended to limit the disclosure so further described in such appended claims.

What is claimed:

1. A signaling device for sensing and indicating the presence of a body exudate in an absorbent article, the device comprising:
a housing; and
a paired IR generator/detector disposed within the housing, the paired IR generator/detector adapted to sense a change in infrared light reflectance due to an insult to the absorbent article, the paired IR generator/detector including an infrared-generating light-emitting diode and an infrared-detecting phototransistor.

2. The device of claim 1, further comprising an attachment mechanism for removably attaching the device to the absorbent article.

3. The device of claim 1, wherein the infrared-generating light-emitting diode generates infrared light with a wavelength of 800 to 980 nanometers.

4. The device of claim 1, wherein the infrared-generating light-emitting diode has been selected to generate a wavelength tuned with a body exudate.

5. The device of claim 1, wherein the housing is flexible.

6. The device of claim 1, wherein the signaling device is adapted to provide notification of the presence of a body exudate in the absorbent article.

7. The device of claim 6, wherein the signaling device is adapted to provide a visual notification.

8. The device of claim 6, wherein the signaling device is adapted to provide an audio notification.

9. The device of claim 6, wherein the signaling device is adapted to provide a wireless notification.

10. The device of claim 6, wherein the signaling device is adapted to provide a tactile notification.

11. The device of claim 10, wherein the tactile notification is a vibratory notification.

12. The device of claim 1, wherein the signaling device is adapted to be held adjacent the absorbent article.

13. The device of claim 1, wherein the signaling device is configured to become activated only when no substantial changes within the interior of the article occur for a period of about five minutes.

14. The device of claim 1, wherein the signaling device is configured to become activated only once no substantial changes within the interior of the article occur for a period of about 20 minutes.

15. A body exudate collection and detection system comprising:
an absorbent article; and
a signaling device adapted to be used in conjunction with the absorbent article for detecting the presence of a body exudate therein, the signaling device including a housing and a paired IR generator/detector disposed within the housing, the paired IR generator/detector including an infrared-generating light-emitting diode and an infrared-detecting phototransistor.

16. The system of claim 15, further comprising an attachment mechanism for removably attaching the signaling device to the absorbent article.

17. The device of claim 15, wherein the signaling device is adapted to provide notification of the presence of a body exudate in the absorbent article.

18. The device of claim 15, wherein the device is adapted to be held adjacent the absorbent article.

19. A signaling device for sensing and indicating the presence of a body exudate in an absorbent article, the device comprising:
a housing;
an attachment mechanism for removably attaching the housing to the absorbent article; and
a paired IR generator/detector disposed within the housing, the paired IR generator/detector including an infrared-generating light-emitting diode and an infrared-detecting phototransistor,
wherein the signaling device is adapted to emit a signal when an insult within the absorbent structure is sensed by the paired IR generator/detector.

20. The signaling device of claim 19, wherein the signaling device includes a plurality of settings that are selected by the user depending upon at least one specification of the absorbent article.

21. The signaling device of claim 19, wherein the signaling device is configured to differentiate between a first insult of the absorbent article with a body fluid from a second insult of the absorbent article with a body fluid.

22. The signaling device of claim 19, wherein the signaling device is configured to discern a steady state environment within the absorbent article after the article is donned before being configured to emit a signal by the signaling device.
